(19) United States
(12) Patent Application Publication
(10) Pub. No.: US 2024/0238316 A1
(43) Pub. Date: Jul. 18, 2024

(54) CANNABINOID POUCH

(71) Applicant: Nirajkumar Karneshbhai PATEL, West Melbourne, FL (US)

(72) Inventor: Nirajkumar Karneshbhai PATEL, West Melbourne, FL (US)

(21) Appl. No.: 18/559,293

(22) PCT Filed: May 10, 2022
(86) PCT No.: PCT/US2022/072235

§ 371 Date: Nov. 6, 2023

Related U.S. Application Data
(60) Provisional application No. 63/186,685, filed on May 10, 2021.

(51) Int. Cl. A61K 31/00; A61K 9/00; A61K 47/10; A61K 47/12; A61K 47/38

(52) U.S. Cl. CPC A61K 31/658; A61K 9/006; A61K 47/10; A61K 47/12; A61K 47/38

(57) ABSTRACT

The disclosure provides oral pouch cannabinoid formulations comprising cannabinoid, an orally acceptable acid, an orally acceptable alcohol, flavor components, and an orally acceptable binder in a water-permeable, water-insoluble pouch, together with methods of making and using the same.

### CROSS-REFERENCE TO RELATED APPLICATIONS

This application claims priority to and the benefit of U.S. Provisional Application Ser. No. 63/186,685, filed on May 10, 2021, which is incorporated herein by reference in its entirety.

### FIELD

This disclosure relates to oral pouch products comprising one or more cannabinoids, e.g., cannabidiol (CBD), tetrahydrocannabinol (THC), or combinations thereof, together with methods of making and using the same.

### BACKGROUND

Cannabinoids, e.g., cannabidiol (CBD), tetrahydrocannabinol (THC), or combinations thereof, have been widely reported to have utility in treating or mitigating a variety of conditions, including epilepsy; nausea and vomiting associated with cancer chemotherapy; loss of appetite and weight loss associated with HIV/AIDS; chronic pain; chronic inflammatory conditions including multiple sclerosis and rheumatoid arthritis; anxiety; depression; and insomnia. Common means for delivery of cannabinoids include smoking cannabis plant materials, inhaling vapor produced by volatilization of liquids containing cannabinoids (vaping), and ingesting food containing cannabinoids. Cannabinoids, however, present some formulation challenges, e.g., poor solubility, unpleasant taste, and variable absorption. Cannabinoids are relatively slowly absorbed when ingested, and while they are rapidly absorbed through the lungs when smoked or vaped, these routes of administration could potentially damage the lungs. Products which deliver cannabinoids via the oral mucosa provide rapid absorption, for example sublingual sprays or tinctures, but face challenges in manufacturing, shelf stability, efficient cannabinoid delivery, taste, and consumer acceptance.

There is a need for cannabinoid products which deliver the cannabinoids across the oral mucosa quickly and efficiently, have good flavor and mouthfeel, and which are stable for long-term storage.

### SUMMARY

The disclosure provides an oral pouch product, comprising one or more cannabinoids, e.g., CBD, THC, or combinations thereof, an organic acid, e.g. benzoic acid; flavor; and binder, in a water-permeable, water-insoluble pouch, wherein the cannabinoids and flavor are released when the pouch is gently chewed or held in the mouth, e.g., between the cheek and gum, and wherein the product provides acceptable cannabinoid release, flavor, mouthfeel, and shelf-stability.

For example, in one embodiment, the disclosure provides a cannabinoid formulation comprising a cannabinoid (e.g., CBD or THC or combinations thereof), an orally acceptable organic acid (e.g. benzoic acid), an orally acceptable alcohol (e.g. propylene glycol), flavor components, and binder in a water-permeable, water-insoluble pouch.

In another embodiment, the disclosure provides a method for making a pouch formulation comprising a cannabinoid (e.g., CBD or THC or combinations thereof), an orally acceptable organic acid (e.g. benzoic acid), propylene glycol, flavor components, and binder, wherein the product is formulated in nonaqueous conditions, using propylene glycol as solvent, and products made thereby.

In another embodiment, the disclosure provides a method of delivering a cannabinoid or a method of treating a condition responsive to cannabinoid therapy, e.g., treating or mitigating a condition selected from epilepsy; nausea and vomiting associated with cancer chemotherapy; loss of appetite and weight loss associated with HIV/AIDS; chronic pain; chronic inflammatory conditions including multiple sclerosis and rheumatoid arthritis; anxiety; depression; and insomnia, comprising administering the above-described pouch to a subject in need thereof.

Further areas of applicability of the present disclosure will become apparent from the detailed description provided hereinafter. It should be understood that the detailed description and specific examples, while indicating certain preferred embodiments of the disclosure, are intended for purposes of illustration only and are not intended to limit the scope of the disclosure.

### DETAILED DESCRIPTION

The formulations of the disclosure are non-aqueous formulations, comprising a cannabinoid (e.g., CBD or THC or combinations thereof), an orally acceptable acid (e.g. an aromatic acid, for example benzoic acid), an orally acceptable alcohol (e.g. propylene glycol), flavor components, and binder, in a water-permeable, water-insoluble pouch. Alcohols, such as propylene glycol, are organic solvents and have a much lower dielectric constant than water. Consequently, the cannabinoid is soluble in the alcohol, and the orally acceptable acid dissolved in the alcohol does not produce ions to the same extent as it would in water. It is found that the cannabinoid in combination with the organic acid is stable relative to cannabinoid without the acid, and moreover provides a better mouth feel and cannabinoid delivery than a cannabinoid in aqueous solution or emulsion.

In a first embodiment, the disclosure provides a cannabinoid pouch formulation comprising a cannabinoid (e.g., CBD or THC or combinations thereof), an orally acceptable acid (e.g. an aromatic acid, for example benzoic acid), an orally acceptable alcohol (e.g. propylene glycol), flavor components, and binder, in a water-permeable, water-insoluble pouch (Formulation 1). For example, the disclosure provides the following formulations:

1.1. Formulation 1 wherein the cannabinoid is selected from CBD, THC and combinations thereof.

1.2. Any foregoing formulation wherein the cannabinoid comprises CBD.

1.3. Any foregoing formulation wherein the cannabinoid comprises THC.

1.4. Any foregoing formulation wherein the cannabinoid comprises delta-8 THC.

1.5. Any foregoing formulation wherein the orally acceptable acid is selected from hydrochloric acid, tartaric acid, pyruvic acid, phosphoric acid, salicylic acid, malic acid, carbonic acid, acetic acid, citric acid, tartaric acid, folic acid, fumaric acid, lactic acid, ascorbic acid, benzoic acid, and combinations thereof; e.g., benzoic acid.

1.6. Any foregoing formulation wherein the orally acceptable acid is an organic acid.

1.7. Any foregoing formulation wherein the orally acceptable acid is an aromatic acid, e.g., benzoic acid, gallic acid, or salicylic acid.

1.8. Any foregoing formulation wherein the orally acceptable acid is benzoic acid.

1.9. Any foregoing formulation wherein the orally acceptable alcohol is propylene glycol.

1.10. Any foregoing formulation wherein the binder is selected from polysaccharides, polyols, sugars, natural fibers, microcrystalline cellulose, cellulose and cellulose derivatives, and mixtures thereof.

1.11. Any foregoing formulation wherein the binder is a hydroscopic but insoluble material.

1.12. Any foregoing formulation wherein the binder comprises microcrystalline cellulose.

1.13. Any foregoing formulation wherein the water-permeable, water insoluble pouch is made of a semi-permeable material which substantially prevents the binder from leaving the bag but permits saliva and therein dissolved components from the powder in the pouch to freely pass through said material.

1.14. Any foregoing formulation wherein the water-permeable, water insoluble pouch is made from one or more polymers or fibers safe for oral use, e.g., selected from polypropylene, low density polyethylene, polyethylene terephthalate, polyurethane, polyvinyl acetate, polyvinyl alcohol, polystyrene, poly(ethylene-vinyl acetate), rayon, silk, cotton, polyester, cellulosic materials (e.g., hydroxypropyl cellulose), and combinations thereof.

1.15. Any foregoing formulation further comprising one or more additional components selected from antioxidants, emulsifiers, preservatives, and solvents.

1.16. Any foregoing formulation further comprising a neutral, orally acceptable mineral salt, e.g., selected from sodium chloride, potassium chloride, and mixtures thereof, e.g., comprising potassium chloride.

1.17. Any foregoing formulation which is substantially free of any active ingredient other than cannabinoid.

1.18. Any foregoing formulation which is substantially free of water.

1.19. Any foregoing formulation which is made under substantially water-free conditions.

1.20. Any foregoing formulation, wherein the contents of the water-permeable, water-insoluble pouch have a pH of less than 7, e.g., pH 5.5 to pH 6.9, e.g., about pH 6.5, when measured in a 10% slurry in water.

1.21. Any foregoing formulation, wherein the ratio by weight of (i) binder and (if present) neutral orally acceptable mineral salt, e.g., potassium chloride, to (ii) cannabinoid, orally acceptable alcohol, and flavor, is from 70:30 from 50:50, e.g., 65:35 to 55:45, e.g. about 60:40.

1.22. Any foregoing formulation wherein the cannabinoid is CBD, the orally acceptable acid is benzoic acid, the orally acceptable alcohol is propylene glycol, and the orally acceptable binder is microcrystalline cellulose.

1.23. Any foregoing formulation wherein the contents of the water-permeable, water-insoluble pouch comprise CBD, benzoic acid, potassium chloride, propylene glycol, flavor, and binder.

1.24. Any foregoing formulation wherein the contents of the water-permeable, water-insoluble pouch comprise cannabinoid, benzoic acid, potassium chloride, propylene glycol, flavor, and microcrystalline cellulose.

1.25. Any foregoing formulation wherein the flavor components are pH neutral.

1.26. Any foregoing formulation wherein the flavor components comprise one or more flavors selected from mint oil, menthol, watermelon, blueberry, pomegranate, strawberry, blueberry, dragonfruit, and cucumber flavors.

1.27. Any foregoing formulation wherein the flavor components comprise mint oil and/or menthol.

1.28. Any foregoing formulation wherein the cannabinoid comprises synthetic cannabinoid.

1.29. Any foregoing formulation wherein the cannabinoid comprises cannabinoid from Cannabis, e.g., Cannabis sativa, Cannabis indica, or Cannabis ruderalis.

1.30. Any foregoing formulation wherein the cannabinoid comprises an extract from Cannabis, e.g., Cannabis sativa, Cannabis indica, or Cannabis ruderalis.

1.31. Any foregoing formulation wherein the cannabinoid comprises CBD but is substantially free of THC, e.g., wherein the cannabinoid comprises less than 1% THC by weight of the cannabinoid.

1.32. Any foregoing formulation wherein the contents of the water-permeable, water-insoluble pouch are in powder form.

1.33. Any foregoing formulation comprising 1%-9%, e.g. 2-5%, e.g., 2.5-3.5%, of cannabinoid and orally acceptable acid, by weight of the contents of the water-permeable, water-insoluble pouch.

1.34. Any foregoing formulation comprising 4-20%, e.g., 5%-15%, e.g., about 5%, or or about 10%, or about 15%, of orally acceptable alcohol, e.g. propylene glycol, by weight of the contents of the water-permeable, water-insoluble pouch.

1.35. Any foregoing formulation comprising 50%-70%, e.g. 55%-60%, of binder, e.g., microcrystalline cellulose, by weight of the contents of the water-permeable, water-insoluble pouch.

1.36. Any foregoing formulation comprising 0.5%-2%, e.g., 1%-1.5%, of orally-acceptable mineral salt, e.g. potassium chloride, by weight of the contents of the water-permeable, water-insoluble pouch.

1.37. Any foregoing formulation wherein the contents of the water-permeable, water-insoluble pouch comprise:
Cannabinoid: 1% to 6%, e.g. 1% to 3%, e.g., 2%
Benzoic acid: 0.5% to 3%, e.g., 0.5% to 1.5%, e.g., 1%
Propylene glycol: 5% to 20%
Flavor: 20% to 35%
Microcrystalline cellulose: 55% to 60%
Potassium chloride: 1% to 1.5%
   wherein all amounts are by weight of the contents of the pouch.

1.38. Formulation 1.35 wherein the weight percent of the solid components comprising microcrystalline cellulose and potassium fluoride is 55% to 65%, e.g., about 60%; and the weight percent of the liquid components comprising propylene glycol, cannabinoid, benzoic acid and flavor is 35% to 45%, e.g., about 40%.

1.39. Formulation 1.35 or 1.36 wherein the weight ratio of cannabinoid to benzoic acid is 1:1 to 3:1, e.g., about 2:1.

1.40. Formulation 1.35, 1.36 or 1.37 wherein the molar ratio of cannabinoid to benzoic acid is 3:1 to 1:3, e.g., from 2:1 to 1:1, e.g., is about 3:2.

1.41. The formulation of any foregoing claim wherein each pouch contains a dose of 1 mg to 250 mg cannabinoid; e.g., 20 mg to 200 mg CBD, or 1 mg to 100 mg THC.

1.42. Any foregoing formulation wherein each pouch contains CBD in the amount of 10 mg to 250 mg, e.g., 20 mg to 200 mg, e.g., 40-60 mg, e.g., about 50 mg.

1.43. Any foregoing formulation wherein each pouch contains THC in the amount of 1 mg to 100 mg, e.g., 1 mg to 20 mg, or 5 mg to 30 mg, e.g., about 10 mg.

1.44. Any foregoing formulation wherein the weight of the contents of the pouch is from 0.2 g to 1 g, e.g. from 0.3 g to 0.5 g, e.g., about 0.4 g.

1.45. Any foregoing formulation when made according to any of Methods 1, et seq.

1.46. Any foregoing formulation for use in a method of delivering cannabinoid to a subject, e.g. for use in a method of treating a condition which may be treated or alleviated with a cannabinoid, e.g., a condition selected from one or more of epilepsy; nausea and vomiting associated with cancer chemotherapy; loss of appetite and weight loss associated with HIV/AIDS; chronic pain; chronic inflammatory conditions including multiple sclerosis and rheumatoid arthritis; anxiety; depression; and insomnia.

1.47. Any foregoing formulation wherein the formulation has a shelf life of at least 6 months, e.g. at temperatures up to 40° C.

1.48. Any foregoing formulation wherein the formulation has a pH of less than 7, e.g., pH 5.5 to pH 6.9, e.g., about pH 6.5, when measured in a 10% slurry in water, and exhibits improved stability, mouth feel, and/or flavor relative to a product having a pH of at least 8 when measured in a 10% slurry in water.

In another embodiment, the disclosure provides a method (Method 1) of making a chewable cannabinoid formulation, e.g., according to any of Formulation 1 above, comprising the steps of
a) dissolving a cannabinoid and an orally acceptable acid in a non-aqueous orally acceptable solvent,
b) adding flavor components,
c) mixing with solid orally acceptable binder and optionally a salt, and
d) filling a water-permeable, water-insoluble pouch with the mixture thus obtained.

For example, Method 1 comprises

1.1. Method 1 wherein the cannabinoid is selected from CBD, THC and combinations thereof.

1.2. Any foregoing method wherein the cannabinoid comprises CBD.

1.3. Any foregoing method wherein the cannabinoid comprises THC.

1.4. Any foregoing method wherein the cannabinoid comprises delta-8 THC.

1.5. Any foregoing method wherein the orally acceptable acid is selected from hydrochloric acid, tartaric acid, pyruvic acid, phosphoric acid, salicylic acid, malic acid, carbonic acid, acetic acid, citric acid, tartaric acid, folic acid, fumaric acid, lactic acid, and benzoic acids; e.g., benzoic acid.

1.6. Any foregoing method wherein the orally acceptable acid is an aromatic acid.

1.7. Any foregoing method wherein the orally acceptable acid is benzoic acid.

1.8. Any foregoing method wherein the orally acceptable alcohol is propylene glycol.

1.9. Any foregoing method wherein the binder is selected from polysaccharides, polyols, sugars, natural fibers, microcrystalline cellulose, cellulose and cellulose derivatives, and mixtures thereof.

1.10. Any foregoing method wherein the orally acceptable acid is selected from hydrochloric acid, tartaric acid, pyruvic acid, phosphoric acid, salicylic acid, malic acid, carbonic acid, acetic acid, citric acid, tartaric acid, folic acid, fumaric acid, lactic acid, and benzoic acids.

1.11. Any foregoing method wherein the orally acceptable acid is an organic acid.

1.12. Any foregoing method wherein the orally acceptable acid is a monoprotic organic acid.

1.13. Any foregoing method wherein the orally acceptable acid is benzoic acid.

1.14. Any foregoing method wherein the molar ratio between cannabinoid and orally acceptable acid in step a) is from 3:1 to 1:3, e.g., from 2:1 to 1:1.

1.15. Any foregoing method wherein the molar ratio between cannabinoid and orally acceptable acid in step a) is about 3:2.

1.16. Any foregoing method wherein the orally acceptable acid is benzoic acid, and the weight ratio of weight ratio of cannabinoid to benzoic acid in step a) is 1:1 to 3:1, e.g., about 2:1.

1.17. Any foregoing method wherein each pouch contains CBD in the amount of 10 mg to 250 mg, e.g., 20 mg to 200 mg, e.g., 40-60 mg, e.g., about 50 mg.

1.18. Any foregoing method wherein each pouch contains THC in the amount of 1 mg to 100 mg, e.g., 5 mg to 30 mg, e.g., about 10 mg.

1.19. Any foregoing method wherein the nonaqueous solvent comprises propylene glycol, e.g. wherein the nonaqueous solvent is propylene glycol.

1.20. Any foregoing method wherein the nonaqueous solvent is heated to facilitate dissolution of the cannabinoid and the orally acceptable acid.

1.21. Any foregoing method wherein the binder is selected from polysaccharides, polyols, sugars, natural fibers, microcrystalline cellulose, cellulose and cellulose derivatives, and mixtures thereof.

1.22. Any foregoing method wherein the binder is a hydroscopic but insoluble material.

1.23. Any foregoing method wherein the binder comprises microcrystalline cellulose.

1.24. Any foregoing method wherein the water-permeable, water insoluble pouch is made of a semi-permeable material which substantially prevents the binder from leaving the bag but permits saliva and therein dissolved components from the powder in the pouch to freely pass through said material.

1.25. Any foregoing method wherein the water-permeable, water insoluble pouch is made from one or more polymers or fibers safe for oral use, e.g., selected from polypropylene, low density polyethylene, polyethylene terephthalate, polyurethane, polyvinyl acetate, polyvinyl alcohol, polystyrene, poly(ethylene-vinyl acetate), rayon, silk, cotton, polyester, cellulosic materials (e.g., hydroxypropyl cellulose), and combinations thereof.

1.26. Any foregoing method further comprising adding one or more additional components selected from antioxidants, emulsifiers, preservatives, and solvents.

1.27. Any foregoing method further comprising adding to the binder a neutral alkali salt, e.g., selected from sodium chloride, potassium chloride, and mixtures thereof, e.g., potassium chloride.

1.28. Any foregoing method wherein no active ingredient is added other than cannabinoid.

1.29. Any foregoing method wherein all steps are carried out under substantially water-free conditions.

1.30. Any foregoing method wherein the product of step b) has an apparent pH of 5 to less than 7, e.g. about 5.5-6.5, when measured using a pH-sensitive glass electrode concentrically surrounded by a reference electrode filled with reference electrolyte, which measures the H⁺ ion concentration of the solution.

1.31. Any foregoing method, wherein the product of step c) has a pH of less than 7, e.g., pH 5.5 to pH 6.9, e.g., about pH 6.5, in a 10% slurry in water.

1.32. Any foregoing method, wherein the ratio by weight of (i) binder and (if present) neutral alkali salt, e.g., potassium chloride, to (ii) cannabinoid, benzoic acid, propylene glycol, and flavor in the product of step c) is from 70:30 from 50:50, e.g., 65:35 to 55:45, e.g. about 60:40.

1.33. Any foregoing method wherein the product of step c) consists of cannabinoid, benzoic acid, potassium chloride, propylene glycol, flavor, and binder.

1.34. Any foregoing method wherein the product of step c) consists of cannabinoid, benzoic acid, potassium chloride, propylene glycol, flavor, and microcrystalline cellulose.

1.35. Any foregoing method wherein the flavor components are pH neutral.

1.36. Any foregoing method wherein the flavor components comprise one or more flavors selected from mint oil, menthol, watermelon, blueberry, pomegranate, strawberry, blueberry, dragonfruit, and cucumber flavors.

1.37. Any foregoing method wherein the flavor components comprise mint oil and/or menthol.

1.38. Any foregoing method wherein the cannabinoid comprises synthetic cannabinoid.

1.39. Any foregoing method wherein the cannabinoid comprises cannabinoid from Cannabis, e.g., Cannabis sativa, Cannabis indica, or Cannabis ruderalis.

1.40. Any foregoing method wherein the product of step c) is in powder form.

1.41. Any foregoing method wherein the product of step c) comprises 2-5%, e.g., 2.5-3.5%, of cannabinoid, by weight.

1.42. Any foregoing method wherein the product of step c) comprises 4-20%, e.g., 5%-15%, e.g., about 5%, or about 10%, or about 15%, of propylene glycol, by weight.

1.43. Any foregoing method wherein the product of step c) comprises 50%-70%, e.g. 55% 60%, of microcrystalline cellulose, by weight.

1.44. Any foregoing method wherein the product of step c) comprises 0.5%-2%, e.g., 1%-1.5%, of potassium chloride, by weight.

1.45. Any foregoing method wherein:
in step a), the amount of cannabinoid is 1% to 3%, e.g., 2%, the orally acceptable acid is benzoic acid in an amount of 0.5% to 1.5%, e.g., 1%, and the non-aqueous orally acceptable solvent is propylene glycol, in an amount of 5% to 20%;
in step b), the amount of flavor components is 20% to 35%; and
in step c), the solid binder is microcrystalline cellulose in the amount of 55% to 60% and a salt is present which is potassium chloride in the amount of 1% to 1.5%;
wherein all amounts are given by weight of the product of step c).

1.46. The foregoing method wherein the weight percent of the microcrystalline cellulose plus the potassium fluoride is 55% to 65% and the weight percent of the propylene glycol plus the flavor is 35% to 40%.

1.47. Any foregoing method wherein in step d), each pouch is filled with a mixture of the product of step c) containing 1 mg to 250 mg cannabinoid, e.g., 20 mg to 200 mg CBD or 1 mg to 100 mg THC.

1.48. Any foregoing method, wherein the product is any of Formula 1, et seq.

In a further embodiment, the disclosure provides a method of delivering cannabinoid to a subject comprising administering an oral pouch cannabinoid formulation according to any of Formulation 1, et. seq. to the subject, e.g., a needed, e.g., up to 3× daily, e.g., wherein the subject suffers from a condition which may be treated or alleviated with a cannabinoid, e.g., wherein the patient suffers from a condition selected from one or more of epilepsy; nausea and vomiting associated with cancer chemotherapy; loss of appetite and weight loss associated with HIV/AIDS; chronic pain; chronic inflammatory conditions including multiple sclerosis and rheumatoid arthritis; anxiety; depression; and insomnia.

### Example 1: Cannabinoid Pouch Formulations

Formulation A-The following components are combined as described above:

| Name of Material | Absolute Quantity(% w/w) |
|---|---|
| CBD | 10.00% |
| Benzoic acid | 2.00% |
| Propylene glycol | 14.40% |
| Flavor Mix | 21.60% |
| MCC | 50.84% |
| Potassium Chloride | 1.16% |
| Total | 100% |

The propylene glycol is heated in a heated mixing tank to 70° C. Benzoic acid is added and mixed until it completely dissolves. Then CBD is added and mixed well. Flavor components are added one by one and mixed to form a liquid premix. Microcrystalline cellulose (MCC) and potassium chloride are mixed in a ribbon blender, then the liquid premix mixture is added to the microcrystalline cellulose (MCC) and potassium chloride in the ribbon blender and all ingredients are mixed well, to form a powder. The powder is then measured to provide the desired dose of cannabinoid and placed in pouches.

Formulation B-The following components are combined as described above:

| Name of Material | Absolute Quantity(% w/w) |
|---|---|
| CBD | 10.00% |
| Benzoic acid | 2.00% |
| Propylene glycol | 4.00% |
| Flavor Mix | 32.00% |
| MCC | 50.84% |
| Potassium Chloride | 1.16% |
| Total | 100% |

Formulation C-The following components are combined:

| Name of Material | Absolute Quantity(% w/w) |
|---|---|
| CBD | 10.00% |
| Benzoic acid | 1.00% |
| Propylene glycol | 5.00% |
| Flavor Mix | 32.00% |
| MCC | 50.84% |
| Potassium Chloride | 1.16% |
| Total | 100% |

Formulation D-The following components are combined as described above:

| | Absolute |
|---|---|
| Name of Material | Quantity(% w/w) |
| CBD | 10.00% |
| Benzoic acid | 1.00% |
| Propylene glycol | 15.40% |
| Flavor Mix | 21.60% |
| MCC | 50.84% |
| Potassium Chloride | 1.16% |
| Total | 100% |

Formulation E-The following components are combined as described above:

| Name of Material | Absolute Quantity(% w/w) |
|---|---|
| CBD | 20.00% |
| Benzoic acid | 1.00% |
| Propylene glycol | 15.40% |
| Flavor Mix | 21.60% |
| MCC | 42% |
| Total | 100% |

Formulation F-The following components are combined as described above:

| Name of Material | Absolute Quantity(% w/w) |
|---|---|
| CBD | 15.00% |
| Benzoic acid | 1.00% |
| Propylene glycol | 5.00% |
| Flavor Mix | 32.00% |
| MCC | 47% |
| Total | 100% |

### Example 2: Cannabinoid Pouch Formulation Performance

The liquid premixes for Formulations C, D, E and F have an apparent pH of about 6.5, while the liquid premixes for Formulations A and B have an apparent pH of about 5.5, when measured using a standard pH sensor (i.e., a pH-sensitive glass electrode concentrically surrounded by a reference electrode filled with reference electrolyte, which measures the H⁺ ion concentration of a solution). Note that while this measurement is not a true pH, as the liquid premixes are non-aqueous, it provides a useful measure of comparative proton activity.

For each of the above formulations, 400 mg of formulation is placed in a water-permeable, water-insoluble pouch, and the pouch containing the formulation is evaluated for taste and organoleptic properties.

Formulations A and B, having higher levels of benzoic acid, are tested and found to have inferior mouth feel relative to Formulations C and D. Formulations C and D provide a “tingling” sensation, which is preferred by users.

Formulations C and D, which have potassium chloride, are found to provide a fresher, cleaner taste compared to Formulations E and F, which do not contain potassium chloride.

Various solvents are tested, including other alcohols. Propylene glycol is preferred over other solvents, as it is found to dissolve both the acid and the cannabinoid efficiently and without excessive heating, which is important, as heating can cause the cannabinoid to volatilize and can generate toxic fumes.

1. An oral pouch cannabinoid formulation comprising a cannabinoid, an orally acceptable acid, an orally acceptable alcohol, flavor components, and an orally acceptable binder in a water-permeable, water-insoluble pouch.

2. The formulation of claim 1 wherein the cannabinoid comprises CBD, THC, or mixtures thereof.

3. The formulation of claim 1 wherein the orally acceptable acid is benzoic acid.

4. The formulation of claim 1 wherein the orally acceptable alcohol is propylene glycol.

5. The formulation of claim 1 wherein the binder comprises microcrystalline cellulose.

6. The formulation of claim 1 further comprising a neutral, orally acceptable mineral salt, e.g., selected from sodium chloride, potassium chloride, and mixtures thereof, e.g., comprising potassium chloride.

7. The formulation of claim 1 which is substantially free of any basic ingredient other than cannabinoid.

8. The formulation of claim 1 which is made under substantially water-free conditions.

9. The formulation of claim 1, wherein the contents of the water-permeable, water-insoluble pouch have a pH of less than 7, e.g., pH 5.5 to pH 6.9, e.g., about pH 6.5, when measured in a 10% slurry in water.

10. The formulation of claim 1, wherein the ratio by weight of
(i) binder and (if present) neutral orally acceptable mineral salt, e.g., potassium chloride, to
(ii) cannabinoid, orally acceptable acid, orally acceptable alcohol, and flavor, is from 70:30 from 50:50, e.g., 65:35 to 55:45, e.g. about 60:40.

11. The formulation of claim 1 wherein the orally acceptable acid benzoic acid, the orally acceptable alcohol is propylene glycol, and the orally acceptable binder is microcrystalline cellulose.

12. The formulation of claim 1 wherein the contents of the water-permeable, water-insoluble pouch are in dry powder form.

13. The formulation of claim 1 comprising
1%-9%, e.g. 2% to 5%, e.g., 2.5% to 3.5%, of cannabinoid and orally acceptable acid;
4% to 20%, e.g., 5% to 15%, e.g., about 5%, or or about 10%, or about 15%, of orally acceptable alcohol, e.g. propylene glycol;
50%-70%, e.g. 55%-60%, of binder, e.g., microcrystalline cellulose;
0.5%-2%, e.g., 1%-1.5%, of orally-acceptable mineral salt, e.g. potassium chloride,
wherein all percentages are by weight of the contents of the water-permeable, water-insoluble pouch.

14. The formulation of claim 1 wherein the contents of the water-permeable, water-insoluble pouch comprise:
(a) Cannabinoid: 1% to 6%, e.g. 1% to 3%, e.g., 2%
(b) Benzoic acid: 0.5% to 3%, e.g., 0.5% to 1.5%, e.g., 1%
(c) Propylene glycol: 5% to 20%
(d) Flavor: 20% to 35%
(e) Microcrystalline cellulose: 55% to 60%
(f) Potassium chloride: 1% to 1.5%
wherein all amounts are by weight of the contents of the water-permeable, water-insoluble pouch.

15. The formulation of claim 14 wherein the weight percent of the solid components comprising microcrystalline cellulose and potassium fluoride is 55% to 65%, e.g., about 60%; and the weight percent of the liquid components comprising propylene glycol, cannabinoid, benzoic acid and flavor is 35% to 45%, e.g., about 40%.

16. The formulation of claim 14 wherein the weight ratio of cannabinoid to benzoic acid is 1:1 to 3:1, e.g., about 2:1.

17. The formulation of claim 1 wherein each pouch contains a dose of 1 mg to 250 mg cannabinoid; e.g., 20 mg to 200 mg CBD, or 1 mg to 30 mg THC.

18. A method of making an oral pouch cannabinoid formulation, e.g., according to claim 1, comprising the steps of
a) dissolving cannabinoid and an orally acceptable acid in a non-aqueous orally acceptable solvent,
b) adding flavor components,
c) mixing with solid binder and optionally a salt, and
d) filling a water-permeable, water-insoluble pouch with the mixture thus obtained.

19. A method of delivering cannabinoid to a subject comprising administering an oral pouch cannabinoid formulation according to claim 1 to the subject.

20. The formulation of claim 15 wherein the weight ratio of cannabinoid to benzoic acid is 1:1 to 3:1, e.g., about 2:1.
